(19) United States
(12) Patent Application Publication
(10) Pub. No.: US 2004/0143343 A1
(43) Pub. Date: Jul. 22, 2004

(54) Post-radical prostatectomy continence implant

(76) Inventor: Joseph A. Grocela, Boston, MA (US)

Correspondence Address: Mark R Shanks, REED SMITH LLP, 1301 K Street NW, Suite 1100 East Tower, Washington, DC, 20005-3373, US

(21) Appl. No.: 10/345,936

(22) Filed: Jan. 17, 2003

(51) Int. Cl. A61F 2/04

(52) U.S. Cl. 623/23.66; 600/30; 128/898

(57) ABSTRACT

A spacer useful for relieving excess pressure exerted on a sphincter muscle by an anatomical structure. A preferred embodiment relates to a urethral spacer. The urethral spacer is a disk-like element formed of an absorbable, biodegradable material, and includes a slot through a portion of the device which permits the disk to be opened up and positioned around the urethra. The urethral spacer is sutured in place around the urethra adjacent to or around the urinary sphincter, preferably after a radical prostatectomy.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The present invention relates broadly to incontinence, and more specifically to treating incontinence due to radical prostatectomy.

2. Description of the Related Art

Radical prostatectomy is the surgical procedure for removing the male prostate gland due to, e.g., prostate cancer. Approximately 50,000 radical prostatectomies are performed in the United States each year. Between 2% to 30% of patients experience urinary incontinence after a radical prostatectomy.

Urinary incontinence is the inability to control emptying of the urinary bladder. Healthy continence involves several pelvic organs, muscles, and tissues. The sphincter muscle is located at the base of the bladder and the proximal portion of the urethra. As the bladder fills, pressure in the urethra is higher than that in the bladder and the sphincter remains closed. The sphincter opens as pressure in the bladder rises and exceeds the intra-urethral pressure. The detrusor, the large smooth muscle of the bladder, then contracts to empty urine.

Once the prostate is removed, the bladder falls against the urinary sphincter and urogenital diaphragm. The drop creates tension in the bladder walls, causing the sphincter to open. As a result, urine is lost at times when the bladder pressure exceeds the pressure in the urethra, such as during a sneeze or cough. Stress urinary incontinence after prostate surgery may be temporary or permanent, depending on the amount of damage to the nerves and blood vessels supplying the bladder and urethra.

Previously known devices have been directed to stress incontinence in males due to sphincter damage occurring during the radical prostatectomy. Particularly, administering collagen injections to the sphincter, implanting an artificial sphincter, or inserting a sling to support the bladder are previously considered methods of treating incontinence. In some cases, a balloon is implanted next to the bladder. However, balloons do not successfully stem urine leakage. Further, balloons are made from silicone, which increases the risk of infection. None of these methods of treating male incontinence address the problem of helping an undamaged sphincter function normally.

U.S. Pat. No. 6,042,534, issued to Gellman et al., (the '534 patent) relates to a stabilization sling for use in minimally invasive pelvic surgery helping female incontinence. In FIG. 1, the '534 patent describes a biocompatible sling 10 for supporting the urethra and bladder neck. The sling 10 has an elongated shape with a central portion 12, a first end portion 14, a second end portion 16, suture receiving sites 18 and a visual indicator 20. The visual indicator 20 helps the surgeon position the sling 10 centrally about the urethra. Sutures are threaded through the receiving sites 18 and secured to bone anchors or stables within the pelvis. Absorbable slings are described that promote tissue growth, and absorb within 3 to 6 months (col. 8, II. 31-39).

U.S. Pat. No. 6,832,214 to Raz, et al., (the '214 patent) relates to a sling for treating male incontinence resulting from urethral sphincter damage. For males, the sling is positioned between the descending rami of the pubic bone and below the urethra to compress the bulbar urethra. The sling is secured to the pubic bone using anchors (col. 2, II. 13-21).

U.S. Pat. No. 5,368,859, issued to Dunn et al., (the '859 patent) relates to a biodegradable system for regenerating the periodontium. The '859 patent addresses using absorbable and biodegradable materials for periodontal restoration.

Although these devices and methods generally functioned well and provided advantages over prior devices, the devices did not provide relief from male incontinence caused by bladder falling after removal of the prostate gland. Further, these devices do not address situations where there is little or no sphincter damage. What is needed is a device for altering the post-radical prostatectomy anatomy and anatomy of other anatomical systems to allow a sphincter to function normally.

### SUMMARY OF THE INVENTION

One aspect of the present invention relates to an implant for treating incontinence in a patient having a urethra, bladder, and a natural sphincter, the implant having a spacer comprising an opening therein sized and configured for accommodating the urethra; and a slot extending from said opening to an outer edge of said spacer, wherein the spacer has bendable portions on opposing sides of the slot that can be pushed forced away from each other creating an open path for disposing the implant around the urethra and between the bladder and the urinary sphincter.

Another aspect of the present invention relates to an implant for treating incontinence including spacing means for spacing a human bladder apart from a human urinary sphincter.

Yet another aspect of the present invention relates to a method of alleviating male incontinence due to radical prostatectomy in a patient having a urethra, bladder, bladder neck, and urinary sphincter, the method including inserting a spacer between the bladder and the urinary sphincter.

Additional aspects and advantages of the invention will be set forth in part in the description which follows and in part will be obvious from the description, or may be learned by practice of the invention.

### BRIEF DESCRIPTION OF THE DRAWINGS

The accompanying drawings, which are incorporated in and constitute a part of this specification illustrate some embodiments of the invention and, together with the description, serve to explain some aspects, advantages, and principles of the invention. In the drawings,

FIGS. 1a and 1b illustrate a first exemplary embodiment of a urethral spacer in accordance with the present invention.

FIG. 2a illustrates a pre-radical prostatectomy anatomy, in a midsagittal sectional view of the male pelvic area.

FIG. 2b illustrates a post-radical prostatectomy anatomy, in a midsagittal sectional view of the male pelvic area.

FIG. 3 illustrates an exemplary embodiment of a urethral spacer implanted in a post-radical prostatectomy anatomy, in a midsagittal sectional view of the male pelvic area.

FIGS. 4a-4e illustrate a method of implanting an embodiment of the present invention, in midsagittal sectional views of the male pelvic area.

FIG. 5a illustrates an alternative embodiment of the present invention.

FIG. 5b illustrates an alternative embodiment implanted, in a midsagittal sectional view of the male pelvic area.

FIG. 6 illustrates alternative uses of the present invention.

### DETAILED DESCRIPTION OF THE PREFERRED EMBODIMENTS

The present invention has been made in view of the above circumstances, one aspect of which is the provision of a device for supporting the male bladder to prevent incontinence. A further aspect of the invention is providing a bioabsorbable device for treating male incontinence. Yet another aspect of the present invention is the provision of bulk between the bladder and the sphincter. Still another aspect the provision of an implant for allowing various sphincters found in the human body to function normally. A further aspect of the present invention is the minimization of the amount of incision and dissection of bodily structure needed when administering to urinary incontinence. Still another aspect of the present invention is altering the male post-radical prostatectomy anatomy.

The invention relates to a spacer for relieving excess pressure exerted on a sphincter muscle by an anatomical structure. A preferred embodiment relates to a urethral spacer. The urethral spacer is a disk-like element formed of an absorbable, biodegradable material, and includes a slot through a portion of the device which permits the disk to be opened up and positioned around the urethra. The urethral spacer is sutured in place around the urethra adjacent to or around the urinary sphincter, preferably after a radical prostatectomy.

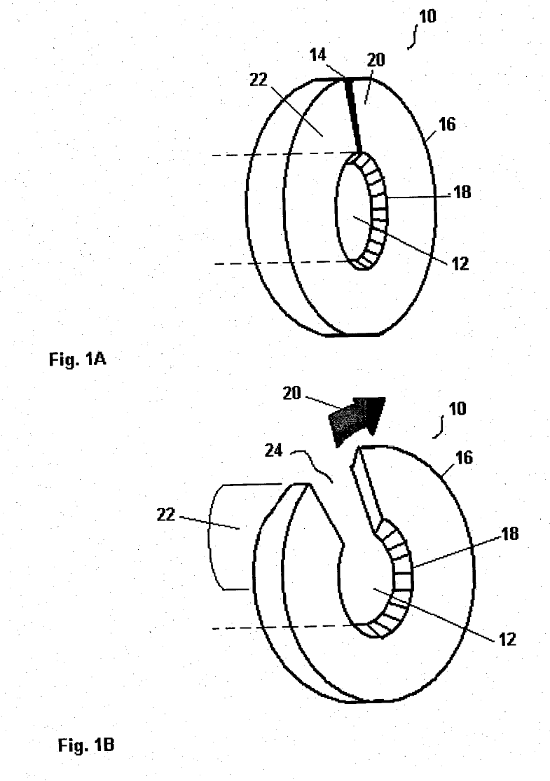

A first exemplary embodiment of the present invention is shown in FIGS. 1a and 1b. The post-radical prostatectomy implant of the present invention includes a spacer 10, preferably having a round, disc-like shape. The spacer 10 includes a circular opening 12 and, in some embodiments, a slot 14. The opening 12 is sized to fit snugly around a urethra. The slot 14 extends from an outer edge 16 of the spacer 10 to an inner edge 18 of the spacer, terminating at the opening 12. Portions of the implant 20, 22, are located on each side of the slot 14. The implant is opened up by pushing or pulling the portions 20, 22 on each side of the slot 14 away from each other, thus, providing an open path 24 to the opening 12. The open spacer 10 can be positioned around a urethra 42 (not shown in FIG. 1), as further described below. Once positioned, the portions 20, 22 on each side of the slot 14 are released and meet at the slot 14, thus closing the spacer 10 around the urethra 42 (not shown in FIG. 1).

The spacer 10 has an outer diameter of approximately 2.0 to 5.0 centimeters. The opening 12 measures between 0.4 centimeters to 1.0 centimeter in diameter, with 0.5 to 0.7 centimeters being, preferably, appropriately sized for typical pediatric use. The spacer 10 has a cylindrical height ranging from approximately 2 millimeters to 1 centimeter.

The spacer 10 is preferably made from a bioabsorbale material, including but not limited to, fascia, dermis, or collagen, further optionally cross-linked, and may further optionally include a material for providing more rigidity, e.g., Dacron. Other suitable bioabsorbable materials that may be used include, but are not limited to, Polydiaxonone Suture (PDS), or Vicryl. The implant primarily provides support, and need only be sufficiently pliable for proper positioning about the urethra 42. The use of a bioabsorbable is preferred for two reasons. First, the material greatly reduces the risk of foreign body reactions, for example, the formation of stones. Second, bioabsorbable material is favored because it promotes natural tissue growth, re-structuring the anatomy. Over time, the body connective tissue grows over the spacer 10, providing reinforcing structure to the bladder 40 and urethra 42 (not shown in FIG. 1). The bioabsorbable material decreases as natural tissue increases, leaving natural tissue in place of the spacer 10.

Bioabsorbable materials come from a wide variety of sources. It will be appreciated by those skilled in the art that such materials with sufficient rigidity and flexibility characteristics may be used without departing from the scope of the present invention. It will be further appreciated by one skilled in the art that while bioabsorbable material is preferred, biocompatible, nonabsorbable materials, such as polymers, nylon, silicon, nitinol, surgical stainless steel, and the like may be used without departing from the scope of the present invention.

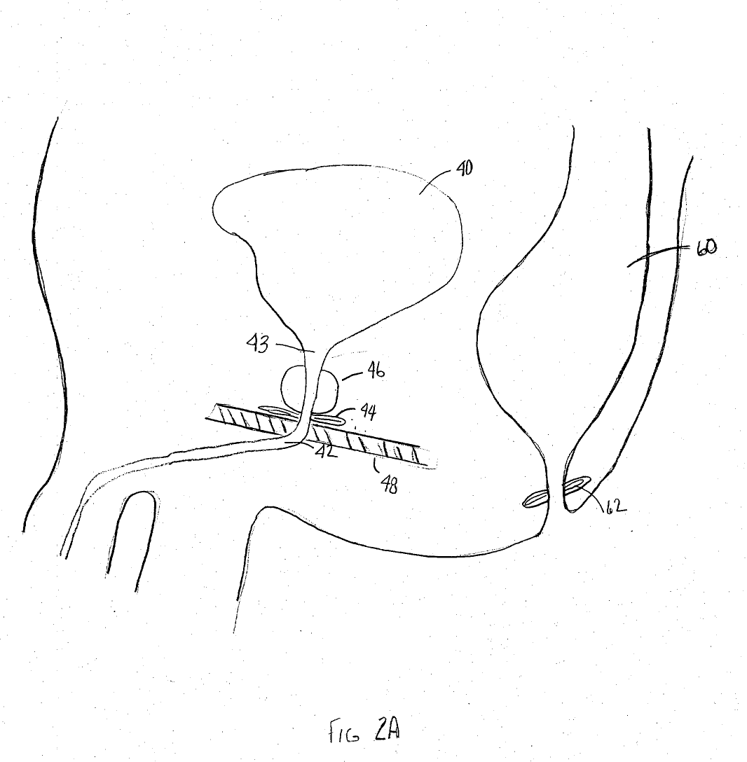

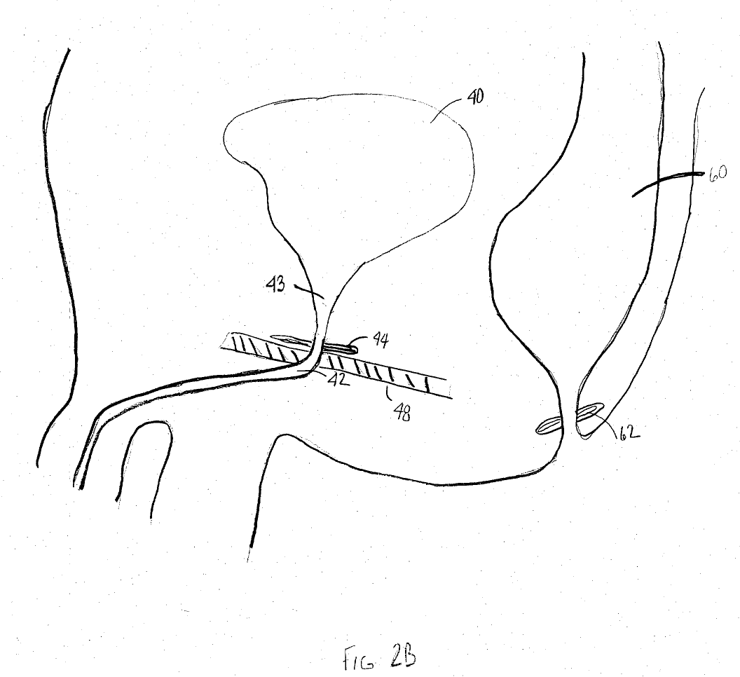

The present invention is further described in light of the anatomy with which it is used. FIG. 2a illustrates the pre-radical prostatectomy anatomy. The bladder 40 holds urine. The urethra 42 allows urine to drain from the bladder 40. The bladder neck 43 is a funnel-shaped, s tissue leading from the bladder 40 to the urethra 42. The urinary sphincter 44 is a muscular ring that controls the opening and closing of the bladder 40. The prostate gland 46 lies underneath the bladder 40 and adjacent to the urethra 42, just above the urinary sphincter 44. The urogenital diaphragm 48 is a thin musculo-fascial sheet providing support to the immediate structures, including the prostate 46, urethra 42, and urinary sphincter 44. The rectum 60 lies anterior to the bladder 40 and urethra 42. The anal sphincter muscle 62 controls the opening and closing of the rectum 60.

FIG. 2b illustrates the post-radical prostatectomy anatomy of a patient after the prostate has been removed. With the prostate removed, the bladder 40 is surgically joined to the urethra 42 and the sphincter 44, asserting added pressure on the urinary sphincter 44 and urogenital diaphragm 48.

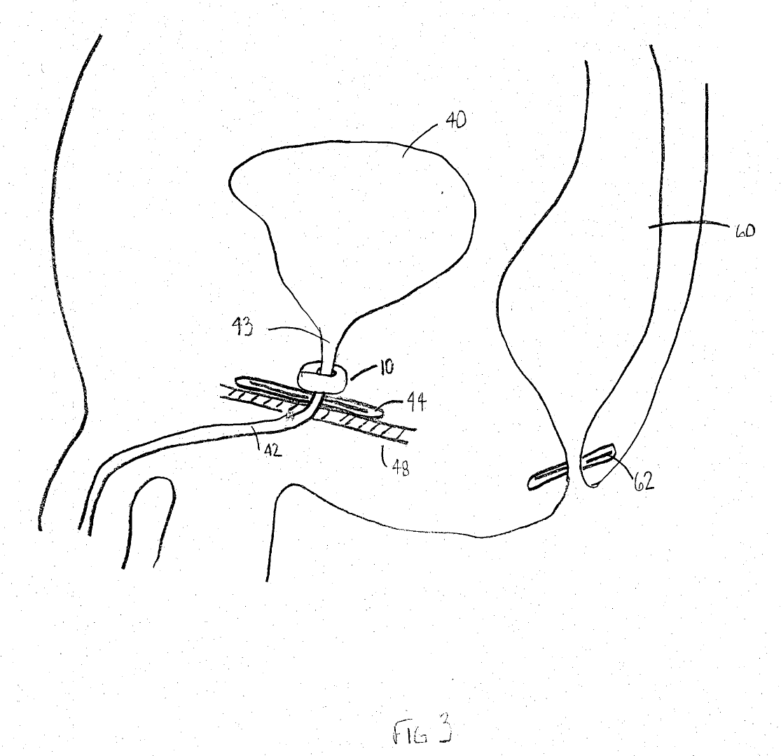

FIG. 3 illustrates an exemplary embodiment of a urethral spacer 10 implanted in a post-radical prostatectomy anatomy, in a midsagittal sectional view of the male pelvic area.

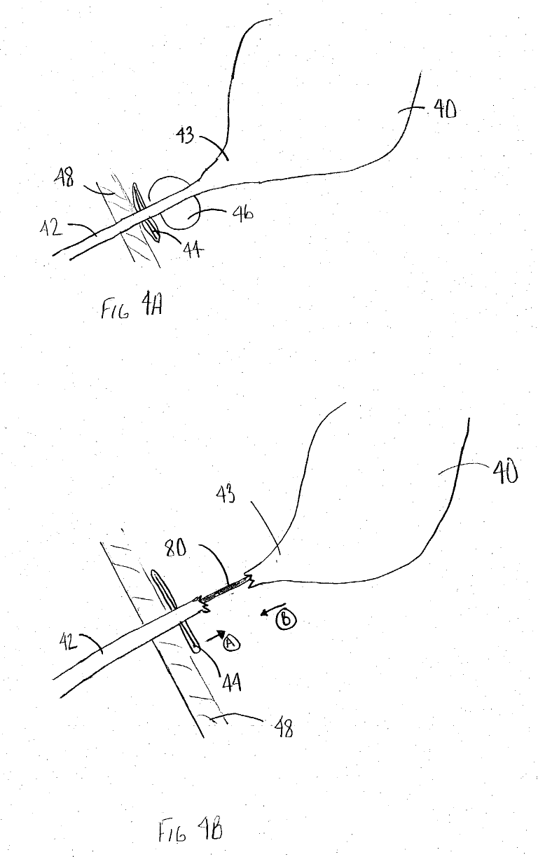

FIGS. 4a through 4e illustrate an exemplary process of inserting a spacer 10 into the post-radical prostatectomy anatomy of a patient. Referring to FIGS. 4a and 4b, a catheter 80, e.g., a Foley-type catheter is extended through the urethra 42 and into the bladder neck 43. The prostate gland 46 is removed, leaving the urethra 42 exposed between the urinary sphincter 44 and bladder neck 43. Portions of the urethra 42, bladder neck 43, urethral sphincter 44, and diaphragm 48 may be damaged during the procedure, potentially causing incontinence, are removed, either with the prostate gland 46, or thereafter. A small portion of the urethra 42 directly above the urinary sphincter 44 usually remains. The remaining portion of the urethra 42 and remaining portion of the bladder neck 43 are pulled together along the arrows A and B, and joined together, e.g. by suturing, to form the anastomosis.

Referring to FIG. 4c, to insert the spacer 10, the implant is opened up by forcing the portions 20, 22 on each side of the slot 14 away from each other, thus, providing an open path 24 to the opening 12. The spacer 10 is moved toward point C, with the urethra 42 in the open path, until the urethra 42 is located in the opening 12, with the spacer 10 surrounding the urethra 42. Once positioned, the portions 20, 22 on each side of the slot 14 are released and at least partially close the slot 14, thus closing the spacer 10 around the urethra 42. The spacer 10 is placed between the bladder 40 and the urinary sphincter 44, preferably in approximately the same position as that previously occupied by the prostate gland 46. The spacer 10 then absorbs pressure exerted by the bladder 40.

Referring to FIG. 4d, once the spacer 10 is in place, sutures, surgical glue, or the like (not shown) may further optionally be used to secure the spacer 10 and relieve absorbed pressure from the bladder 40. Sutures may be secured to the bladder 40, urethra 42, or urogenital diaphragm 48. In FIG. 4d, four suture points 50 are shown. However, it will be appreciated by one skilled in the art that at least two suture points 50 are sufficient to secure the spacer 10. Further, the location of the suture points 50 are illustrative, as suture points 50 can be placed in a variety of locations on the spacer 10 without departing from the scope of the present invention. Additionally, surgical glue or biocompatible staples may be used to secure the spacer 10.

It will be appreciated by one skilled in the art that the spacer 10 may be inserted during the same procedure as the radical prostatectomy. However, the spacer 10 may also be inserted during another procedure, some time after the radical prostatectomy has been performed.

As described with the preferred embodiment, alternative embodiments of devices in accordance with the present invention are preferably made from bioabsorbable material. However, it will be appreciated by one skilled in the art that biocompatible material may be used without departing from the scope of the present invention.

Although the spacer 10 of the present invention has been described as having a disc-like shape, it will be appreciated by one skilled in the art that the over-all shape of the spacer 10 can take a variety of forms. Further, it will also be appreciated by one skilled in the art that the shape of the opening 12 is designed to accommodate a human urethra without squeezing the urethra, and though a generally circular or elliptical shape is preferred, a variety of shapes can serve this purpose.

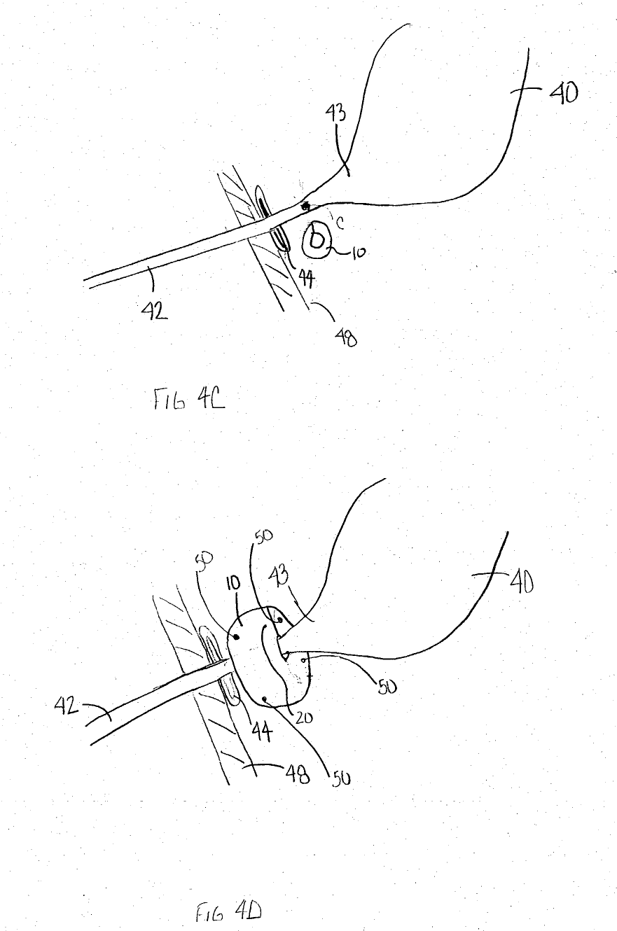

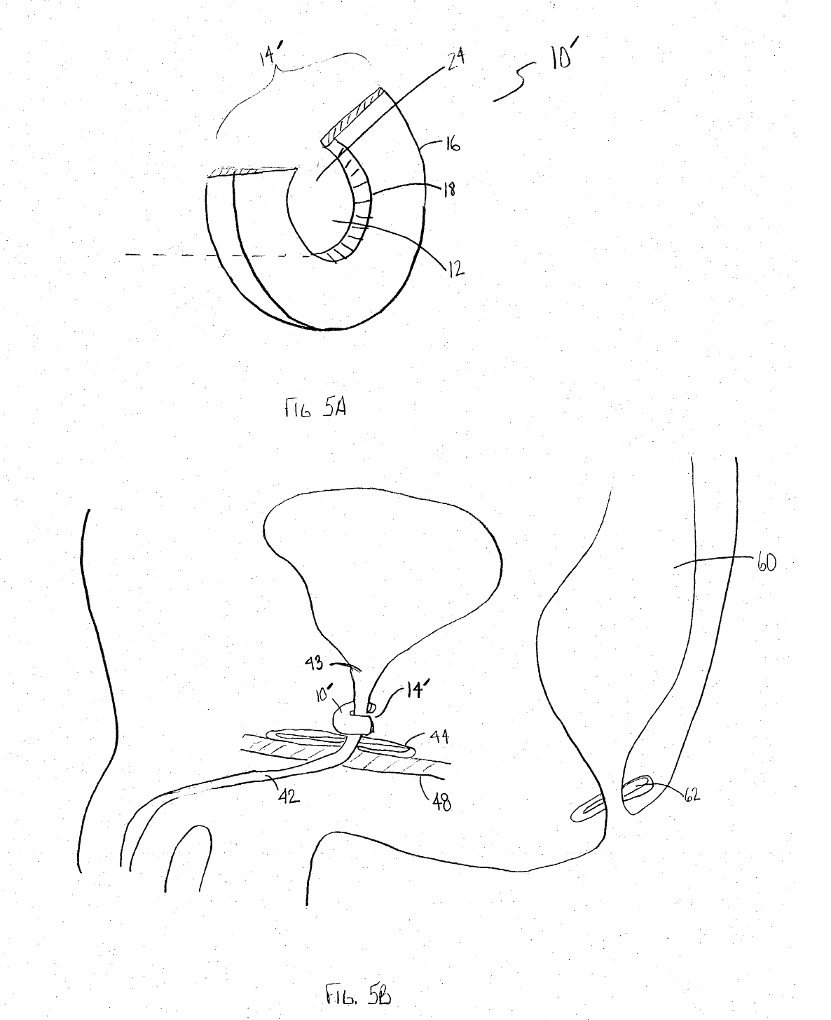

Although the spacer 10 of the present invention have been described as being a closed disc, it will be appreciated by one skilled in the art that the spacer 10 may extend partially around the urethra 42. Referring to FIG. 5a, an alternative embodiment of the present invention, which extends partially around the urethra 42, is illustrated. In this embodiment, the slot 14′ is wider than in the embodiment illustrated in FIGS. 1a, 1b, up to approximately one-half (½), preferably one-third (⅓), of the circumference of the spacer 10′, always leaving an open path. In other words, the spacer 10′ encircles approximately at least one-half (½), and preferably two-third (⅔) of the circumference of the urethra 42.

Referring to FIG. 5b, the alternative embodiment of FIG. 5a is illustrated implanted in the post-radical prostatectomy male anatomy. This embodiment is particularly useful when the spacer 10′ is inserted in a later procedure performed sometime after the radical prostatectomy, i.e., at a time after the surgical opening used to perform the radical prostatectomy has been closed. Particularly, scar tissue may develop after a radical prostatectomy, and usually does so between the urethra and the rectum 60. Thus, the spacer 10′ may vary in thickness, with thicker portions located on the urethral side of the spacer 10′ opposite the slot 14′.

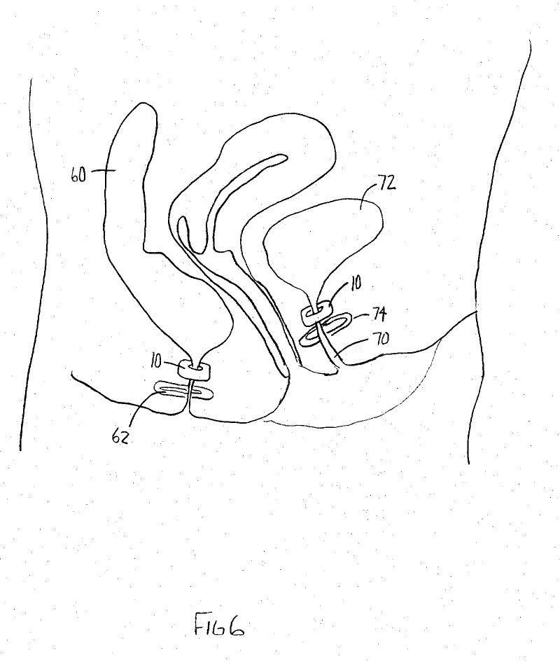

It will also be appreciated by one skilled in the art that the spacer 10 of the present invention may be used to support anatomy preventing the proper function of a sphincter in other parts of the body. Referring to FIG. 6, a midsagittal section view of the female pelvic area is illustrated. Particularly, a rectum 60 is opened and closed by an anal sphincter muscle 62. Fecal incontinence is caused by prolapse of the rectum 60 putting pressure on the anal sphincter 62, causing it to open. A spacer in accordance with the present invention, such as spacer 10 or 10′, may be implanted between the rectum 60 and the anal sphincter muscle 62 to support the rectum 60 and relieve pressure on the anal sphincter muscle 62. Still referring to FIG. 6, the spacer 10 of the present invention may also be used in cases where bladder or pouch neobladder reconstruction is performed. Similar to procedures previously described herein, a spacer in accordance with the present invention, such as spacer 10 or 10′, is placed around the urethra 70 between the bladder or pouch neobladder 72 and the sphincter urethra muscle 74. Generally, the spacer 10 of the present invention is appropriately used whenever anatomical structures are exerting pressure on a sphincter which otherwise operates normally. The spacer 10 is placed above a sphincter and between the sphincter and whichever anatomical structure is exerting excess pressure on the sphincter.

An alternative embodiment of the spacer 10 of the present invention does not include the slot 14 when manufactured. However, at the time of performing the method of implanting the spacer 10, the physician may cut the spacer to form the slot extending from the outer edge 16 of the spacer to the inner edge 18, terminating at the opening 12. For this purpose, the spacer is preferably formed of a material and of a thickness that can be easily cut by the practitioner with surgical scissors or a scalpel. In this manner, the spacer 10 may be custom cut to fit the anatomy as the differences from one patient to another may dictate, and may be formed as a spacer 10, 10′, or similar shape.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents.

What is claimed is:

1. An implant for treating incontinence in a patient having a urethra, bladder, and a natural sphincter, comprising:
a spacer comprising an opening therein sized and configured for accommodating the urethra; and a slot extending from said opening to an outer edge of said spacer;
wherein said spacer has bendable portions on opposing sides of said slot that can be forced away from each other creating an open path for disposing the implant around the urethra and between the bladder and the urinary sphincter.

2. The implant of claim 1 wherein the implant is made from a bioabsorbable material.

3. The implant of claim 2 wherein the material is selected from the group consisting of fascia, dermis, collagen, Polydiaxonone Suture, and Vicryl.

4. The implant of claim 3 wherein the material is cross-linked.

5. The implant of claim 1 wherein the implant is made from a biocompatible material.

6. The implant of claim 5, wherein the material is selected form the group consisting of a polymer, nylon, silicon, nitinol, and surgical stainless steel.

7. The implant of claim 1 wherein said spacer is disc-like in shape.

8. The implant of claim 1 wherein said opening for accommodating the urethra is circular.

9. An implant for treating incontinence comprising:
spacing means for spacing a human anatomical structure apart from a human sphincter and relieving pressure exerted on the sphincter by the anatomical structure.

10. The implant of claim 9 further comprising means for retaining said spacing means between a urinary sphincter and a bladder.

11. An implant according to claim 9 wherein said spacing means is disc-like in shape.

12. An implant according to claim 9 wherein said spacing means comprises a means for at least partially encircling a urethra.

13. An implant according to claim 10 wherein said means for retaining said spacing means is at least one suture.

14. An implant according to claim 9 wherein the spacing means is formed from a sufficiently soft material that may be cut to custom-fit the size of the implant to a patient's anatomy.

15. A method of alleviating male incontinence due to radical prostatectomy in a patient having a urethra, bladder, bladder neck, and urinary sphincter, the method comprising:
inserting a spacer between the bladder and the urinary sphincter.

16. A method according to claim 15 comprising:
inserting sutures for holding said spacer in place.

17. A method according to claim 15 wherein inserting comprises inserting said spacer at least partially surrounding a human urethra.

18. A method according to claim 15, comprising, before inserting the spacer:
inserting a catheter through the urethra and at least partially into the bladder;
removing a portion of the urethra and the bladder neck, leaving remaining portions of the urethra and the bladder neck; and
pulling the remaining portions of the urethra and the bladder neck towards each other; and
joining the remaining portions together.

19. A method according to claim 15, further comprising:
removing the prostate gland prior to inserting the spacer.

20. A method according to claim 19, wherein inserting the spacer comprises inserting the spacer in approximately the same position adjacent to the urethra as where the prostate gland was positioned.

21. An implant according to claim 7 wherein the opening includes a diameter between 0.4 to 1.0 centimeters.

22. An implant according to claim 7 wherein the implant includes an outer diameter between 2.0 to 5.0 centimeters.

23. An implant according to claim 7, wherein the implant includes a cylindrical height between 2 millimeters to 1 centimeter.

24. An implant according to claim 9 wherein said spacing means comprises a means for at least partially encircling at least one-half of a circumference of a urethra.

25. An implant according to claim 9, wherein said spacing means comprises a means for at least partially encircling at least two-thirds of a circumference of a urethra.
